(19) United States
(12) Patent Application Publication
(10) Pub. No.: US 2026/0210848 A1
(43) Pub. Date: Jul. 23, 2026

(54) SYSTEMS FOR ANALYZING A GASEOUS BIOLOGICAL SAMPLE USING TERAHERTZ TIME-DOMAIN SPECTROSCOPY

(71) Applicants: CENTRE NATIONAL DE LA RECHERCHE SCIENTIFIQUE, PARIS (FR); UNIVERSITE DE LILLE, LILLE (FR); UNIVERSITE POLYTECHNIQUE HAUTS-DE-FRANCE, VALENCIENNES (FR)

(72) Inventors: Romain PERETTI, LAMBERSART (FR); Sophie ELIET, OCHTEZEELE (FR); Adrien PILLET, LILLE (FR); Elsa DENAKPO, BOURG-LA-REINE (FR)

(73) Assignees: CENTRE NATIONAL DE LA RECHERCHE SCIENTIFIQUE, PARIS (FR); UNIVERSITE DE LILLE, LILLE (FR); UNIVERSITE POLYTECHNIQUE HAUTS-DE-FRANCE, VALENCIENNES (FR)

(21) Appl. No.: 19/142,451

(22) PCT Filed: Dec. 19, 2023
(86) PCT No.: PCT/EP2023/086707

§ 371 Date: Jun. 23, 2025

(30) Foreign Application Priority Data
Dec. 23, 2022 (FR) 2214437

(51) Int. Cl. G01N 21/3504; A61B 5/00; A61B 5/08; A61B 10/00; G01N 21/3581; G01N 33/497

(52) U.S. Cl. CPC G01N 21/3504; A61B 5/082; A61B 5/7264; A61B 10/00; G01N 21/3581; G01N 33/497; A61B 2010/0087

(57) ABSTRACT

A system for analyzing a gaseous biological sample, the system including a collecting device for collecting the gaseous biological sample; a time-domain spectroscopy measurement device including a gas analysis cell configured to receive the gaseous biological sample and electromagnetic detector configured to detect at least one first sample time trace, each sample time trace resulting from coherent detection of a sample beam arising from the gas analysis cell traversed by a THz excitation beam; a processing unit including a pre-trained machine learning module for detecting at least one state of a subject, the processing unit is configured to calculate a sample estimator on the basis of the at least one sample time trace and to determine from the estimator and using the machine learning module, the at least one state of the subject.

### TECHNICAL FIELD

The present disclosure relates to systems for analyzing a gaseous biological sample using terahertz (THz) time-domain spectroscopy, especially for diagnostic purposes. The present disclosure relates more particularly to systems for analyzing the exhaled air from a patient (human or animal).

### BACKGROUND

The analysis of volatile organic compounds (VOCs) found in the exhaled air (breath) from a patient has many applications, for example, the diagnosis of diseases such as asthma, diabetes or some cancers (see [Ref. 1]).

Two main methodologies are known today to carry out such an analysis.

A first methodology consists of systematically analyzing all volatile organic compounds, for example, using mass spectroscopy (see [Ref. 2]). However, an instrument for mass spectroscopy is expensive.

A second methodology consists of focusing the analysis on one or a few volatile compounds using an “electronic nose”, which is much less expensive but cannot cover all 3500 volatile organic compounds present in breath (see [Ref. 3]).

Recently, it has been demonstrated (see [Ref. 4]) that terahertz (THz) time-domain spectroscopy, and especially by virtue of a super-resolution method as described in [Ref. 4], can be particularly effective for measuring the relative concentrations of hundreds of volatile components in a biological sample. A spectrometer as described in [Ref. 4] thus allows breath analysis with very good sensitivity.

However, the application of such a method to diagnostics is not considered. Indeed, applying the teachings of [Ref. 4] to diagnostics would require knowledge of the spectra of all the components specific to a disease.

[Ref. 5] also proposed, using the terahertz (THz) time-domain spectroscopy, determining spectral lines or combinations of lines of acetone in exhaled air, for diabetic patients and healthy patients, for diagnostic purposes.

To extend the teaching of [Ref.5] to the diagnosis of different diseases, it would therefore be necessary to have a priori knowledge of the spectra of volatile organic compounds, as well as knowledge of the clusters of VOC markers of a given disease. However, this a priori knowledge would be laborious to establish for a large number of diseases.

The present disclosure proposes a system for analyzing a gaseous biological sample and especially a system for analyzing breath, which allows a rapid diagnosis of predefined diseases without a priori knowledge of either the clusters of VOC markers of a given disease, or the spectra of the volatile organic components.

### SUMMARY

In the present disclosure, the term “comprise” means the same as “include”, “contain”, and is inclusive or open-ended and does not exclude other elements not described or represented. Furthermore, in the present disclosure, the term “about” or “substantially” means the same as “having a margin of less than and/or greater than 10%, for example, 5%”, of the respective value.

The present disclosure relates, according to a first aspect, to a system for analyzing a gaseous biological sample comprising:
a collecting device for collecting said gaseous biological sample;
a time-domain spectroscopy measurement device (120) comprising:
a gas analysis cell configured to receive the collected gaseous biological sample;
electromagnetic emission means configured to emit a substantially collimated THz excitation beam into the gas analysis cell;
electromagnetic detection means configured to detect at least one first sample time trace, each sample time trace resulting from a coherent detection of a THz sample beam arising from the gas analysis cell traversed by the THz excitation beam;
a processing unit comprising a pre-trained machine learning module for detecting at least one state of the subject, wherein the processing unit is configured to:
calculate a sample estimator on the basis of said at least one sample time trace;
determine from said estimator and using the machine learning module, said at least one state of the subject.

The THz excitation beam comprises in a known manner (see, for example, [Ref. 4]) electromagnetic pulses emitted with a given period and a spectrum formed by a predetermined frequency comb. The THz sample beam results from the convolution of the THz excitation beam with a function that depends on the characteristic physical parameters of the gaseous biological sample, for example, the absorption coefficient and/or the refractive index of the sample, this function being referred to as sample transfer function.

A coherent detection of the THz sample beam is a detection sensitive to the effect of the gaseous biological sample on the amplitude and the phase of the incident THz excitation beam.

As is well known, such a coherent detection comprises, in exemplary embodiments, generating an ultrashort terahertz pulse (with a wide bandwidth) from an even shorter femtosecond optical pulse, emitted for example, by a Ti-sapphire laser. The optical pulse is first split to provide an optical probe pulse the path length of which is adjusted using an optical delay line. The optical probe pulse illuminates a terahertz detector which is sensitive to the electric field of the resulting THz sample beam at the time when the optical probe pulse is sent to the detector. By varying the length of the traveled path of the optical probe pulse, a time trace is thus measured based on time. The sample response can be calibrated by means of a reference time trace, obtained under the same experimental conditions but, for example, with the sample removed.

The applicants have shown that the system for analyzing a gaseous biological sample according to the first aspect allows a rapid diagnosis of predefined diseases without a priori knowledges of either the clusters of VOC markers of a given disease, or the spectra of the volatile organic components by virtue of processing the sample time traces and not the spectra. This processing is carried out by means of a pre-trained machine learning module, also referred to as a “prediction module” in the present disclosure. The use of such a prediction module further allows, in exemplary embodiments, the module prediction performance to be increased by continuing to train the prediction module with new data acquired during a clinical use of the analysis system.

According to one or more embodiments, the time-domain spectroscopy measurement device is configured to further detect at least one first reference time trace; and said sample estimator is calculated on the basis of said at least one sample time trace and said at least one reference time trace.

According to one or more exemplary embodiments, the detection means are configured to detect a plurality of sample time traces, the sample estimator being calculated on the basis of said plurality of sample time traces.

In some exemplary embodiments, a sample estimator is obtained, for example, from an average of said sample time traces.

According to one or more exemplary embodiments, the machine learning module is pre-trained by means of a convolutional neural network.

According to one or more exemplary embodiments, the collecting device is configured to collect a breath of a patient and comprises a breath collecting tube.

In exemplary embodiments, the collecting device further comprises carbon dioxide detection means configured to detect the carbon dioxide in the collected breath, a first solenoid valve and first solenoid valve control means configured to open the solenoid valve when the detected carbon dioxide exceeds a predetermined threshold value, the collected breath can be then drawn towards the gas analysis cell.

In exemplary embodiments, the collecting device further comprises an intermediate enclosure configured to receive at least one fraction of the collected breath, a second solenoid valve and second solenoid valve control means configured to open said second solenoid valve, the collected breath can be then drawn towards the gas analysis cell and close said second solenoid valve when a pressure in the gas analysis cell reaches a predetermined threshold value.

According to one or more exemplary embodiments, the collecting device is configured to collect a liquid biological sample and comprises production means for producing, from the liquid biological sample, a gaseous biological sample. A liquid biological sample is, for example, a sample of urine, perspiration or saliva.

According to one or more exemplary embodiments, the electromagnetic emission means and the electromagnetic detection means each comprise a terahertz beacon, each of the terahertz beacons comprising an antenna, a deflecting mirror, for example, a parabolic mirror, and a support configured to securely hold said antenna and said deflecting mirror.

It is thus possible to obtain a collimated terahertz beam from a diverging point source, without using a terahertz lens that generates signal losses. The terahertz beam orientation is also facilitated. Especially, it is possible to provide for the antenna and/or for the deflecting mirror a plate that is configured for positioning and/or orientating said antenna or said deflecting mirror, which presents another benefit with respect to the use of a terahertz lens.

According to one or more exemplary embodiments, the system for analyzing a gaseous biological sample further comprises:
a database comprising, for a set of subjects, at least one sample time trace associated with a state of the subject, and
a training module for training the machine learning module from data in the database;
the processing unit being further configured to send said at least one sample time trace generated by the time-domain spectroscopy measurement device to said database.

The database is advantageously stored in electronic form (“cloud”).

Such a system further allows an update of the machine learning module during the clinical use of the analysis system.

### BRIEF DESCRIPTION OF THE FIGURES

Further advantages and features of the invention presented above will become apparent from the detailed description below, provided with reference to the figures in which:

FIG. 1A, an example of a system for analyzing a gaseous biological sample according to the present disclosure;

FIG. 1B, diagrams exemplifying an example of excitation beam temporal shape and an example of an excitation signal spectrum, in an analysis system according to the present disclosure;

FIG. 1C, a diagram exemplifying an example of a time trace measured by means of an analysis system according to the present disclosure;

FIG. 1D, diagrams showing examples of sample and reference time traces;

FIG. 2A, a diagram exemplifying in greater detail a gas analysis cell in a measurement device of an analysis system according to the present disclosure, with a first example of a collecting device;

FIG. 2B, a diagram exemplifying a second example of a collecting device in an analysis system according to the present disclosure;

FIG. 3, diagrams exemplifying an example of a terahertz beacon in an analysis system according to the present disclosure;

FIG. 4, a figure showing in a three-quarter view, a system for analyzing a gaseous biological sample according to the present disclosure, mounted in a rack;

FIG. 5, a block diagram exemplifying different modules of a system for analyzing a gaseous biological sample according to the present disclosure, in an exemplary implementation according to the present disclosure;

FIG. 6, a block diagram exemplifying different modules of an analysis system configured for training the learning module.

In the various embodiments that will be described with reference to the figures, similar or identical elements bear the same references.

### DETAILED DESCRIPTION

In the following detailed description, only certain embodiments are described in detail in order to ensure clarity of disclosure, but these examples are not intended to limit the general scope of the principles underlying the present description.

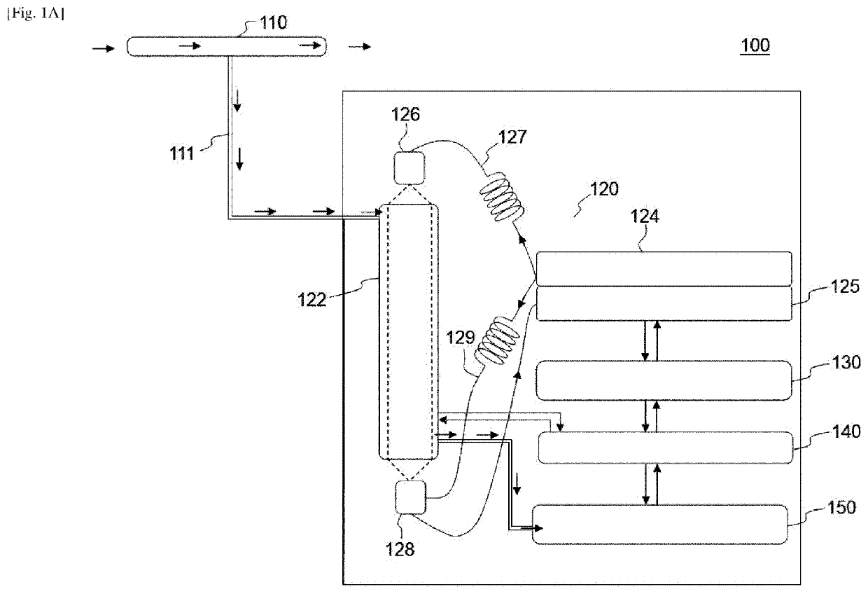

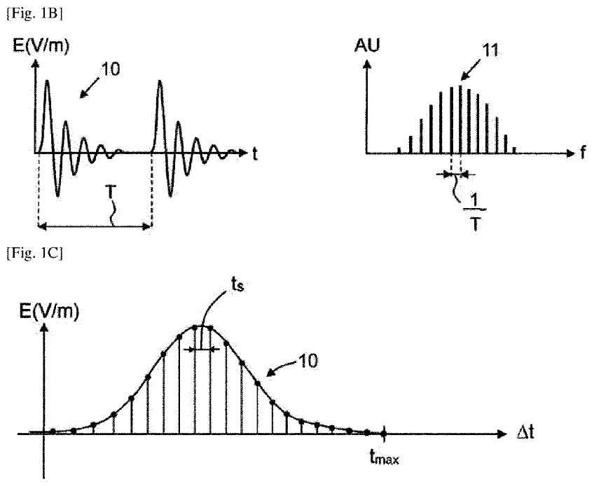

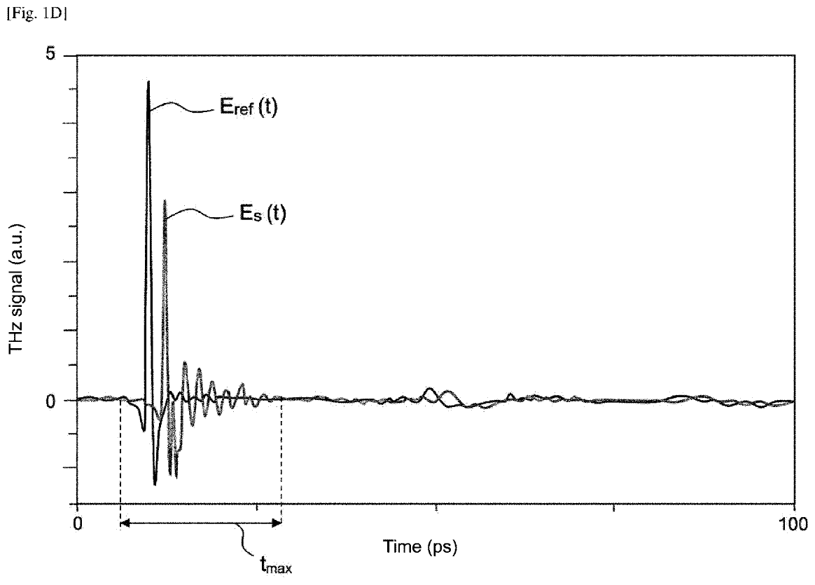

The various embodiments and aspects described in this description can be combined or simplified in many ways. In particular, the steps of the various methods can be repeated, interchanged or run in parallel, unless specified to the contrary.

[FIG. 1A] exemplifies a first example of a system 100 for analyzing a gaseous biological sample according to the present disclosure.

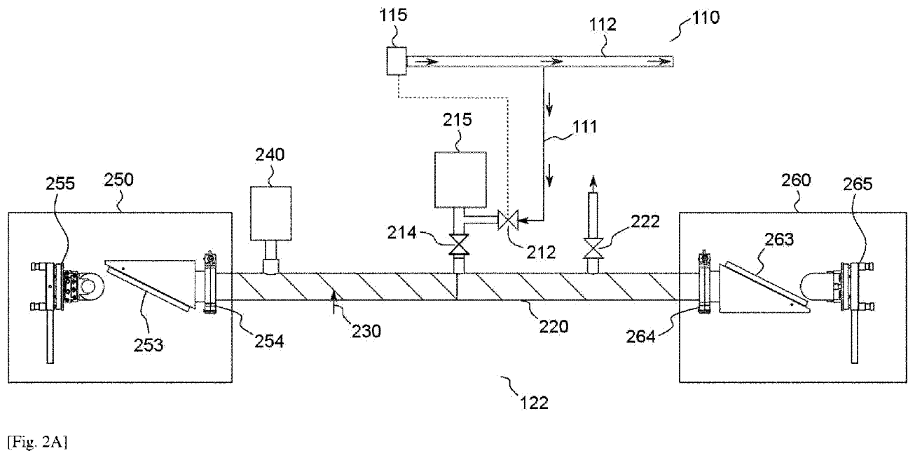

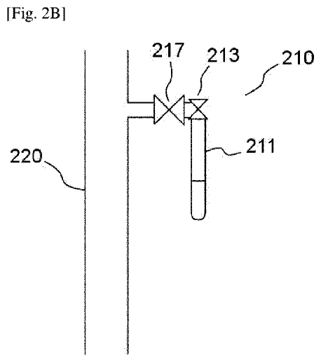

The analysis system comprises a collecting device 110 for collecting the sample and a time-domain spectroscopy (or TDS) measurement device 120. The time-domain spectroscopy device 120 comprises a gas analysis cell 122 configured to receive the collected gaseous biological sample and an example of which will be described in more detail by means of [FIG. 2A]. The time-domain spectroscopy device 120 further comprises electromagnetic emission means 124, 126 configured to emit a substantially collimated THz excitation beam into the gas analysis cell and detection means 128 configured to detect at least one first sample time trace resulting from a coherent detection of a sample beam arising from the gas cell illuminated by the excitation beam. The sample beam results in practice from the convolution of the excitation beam with a transfer function of the gaseous sample to be analyzed. The analysis system 100 also comprises a processing unit 130 comprising a pre-trained machine learning module for the detection of at least one state of a subject. As will be explained in more detail below, the processing unit 130 is configured to calculate an estimator from said at least one sample time trace and determine from said estimator and by means of the machine learning module, said at least one state of the subject. In the example of [FIG. 1A], the analysis system 100 further comprises a vacuum pump 150 configured to evacuate the gas analysis cell 122 via a tube and solenoid valves, as will be described in more detail by means of [FIG. 2A] and a set 140 of microcontroller(s) and/or sensor(s), connected to the vacuum pump 150, the gas analysis cell 122 and the processing unit 130, as will be described in more detail below.

In operation, as illustrated in [FIG. 1A] and according to exemplary embodiments, the gaseous sample passes through the collecting device 110, for example, a collecting device for collecting a breath of a patient; a part of the sample passes through the collecting device and a part is drawn via a bypass capillary 111. The path of the gaseous biological sample is indicated by simple arrows in [FIG. 1A]. The breath collector comprises, for example, a tube, for example, a PTFE tube. The tube presents, for example, a diameter of between about 10 mm and about 15 mm and a length of between about 20 cm and about 40 cm. The tube in the example of the breath collecting device 110 exemplified in [FIG. 1A] comprises one end opened to the open air to allow breath circulation. This prevents, on the one hand, any suction effect on the subject, the cell 122 being under vacuum, and on the other hand, allows the collection in the cell of the part of the breath that is to be analyzed. For example, if the air coming from the bottom of the lungs is to be analyzed, the suction of the breath into the cell can be triggered towards the end of exhalation. On the contrary, if the air coming from the mouth or bronchi is to be analyzed, the aspiration can be started as early as possible in the exhalation.

The capillary 111 opens according to examples into an intermediate enclosure (not shown in [FIG. 1A] but disclosed in more detail in [FIG. 2A]) and then into the gas analysis cell 122 of the time-domain spectroscopy measurement device 120, where it is analyzed.

As previously explained, for the analysis of the gaseous sample, the time-domain spectroscopy device 120 comprises one of the electromagnetic emission/detection means 124, 125, a THz emitter 126 as well as a THz receiver 128.

THz emitters/receivers configured for the emission/reception of pulses in the THz frequency band (that is, between about 0.2 Thz and about 8 Thz) are known to a person skilled in the art.

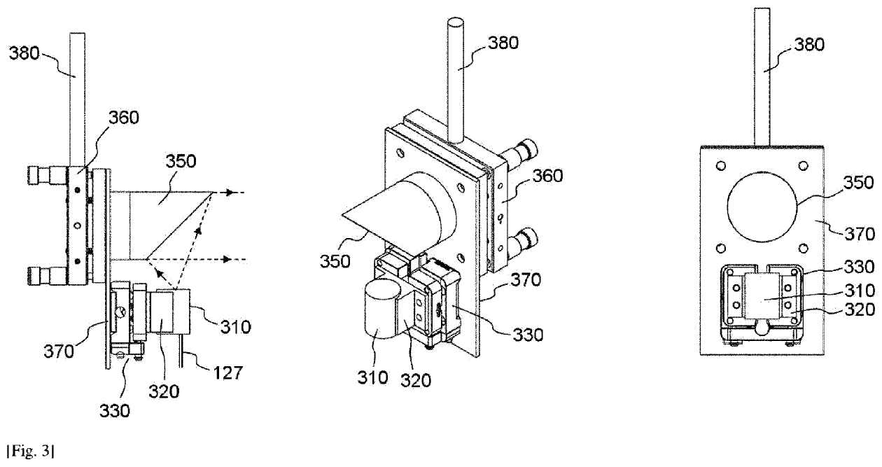

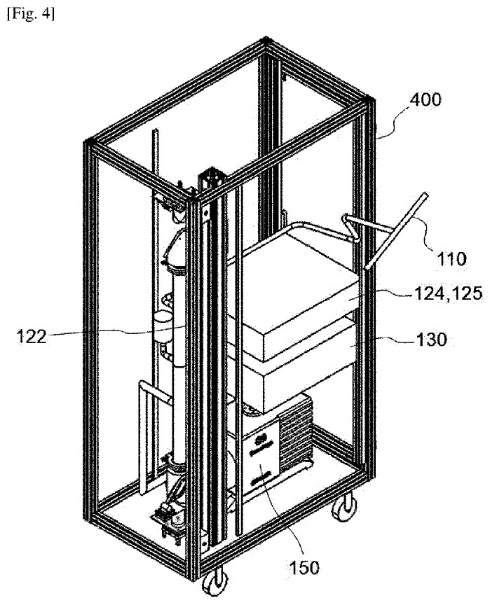

In exemplary embodiments, the emitter 126 and receiver 128, referred to as “THz beacons” in the present disclosure, comprise an antenna and a collimator, for example, a lens or a parabolic mirror. Examples of THz beacons are disclosed in more detail in [FIG. 3].

The electromagnetic emission means 124 comprise, for example, and in a known manner a pulsed femtosecond laser, for example, a Ti-sapphire laser, and a delay line; in operation, a femtosecond pulse is separated into a first pulse directed towards the antenna of the THz emitter 126 and a second pulse, or optical probe pulse, directed towards the antenna of the THz receiver 128 after having undergone a variable time delay by virtue of the delay line.

The femtosecond laser is, for example, a frequency comb pulse laser configured to excite the antenna of the THz beacon 126 (emitter), the femtosecond pulses being routed to the antenna, for example, by an optical fiber 127. The antenna of the THz beacon 126 comprises, for example, a semiconductor element and polarized electrodes. Illuminating the semiconductor element at an energy greater that the band gap energy generates free carriers. The semiconductor thus passes from an insulating state to a conducting state generating an electric current between the polarized electrodes of the antenna. The result is the emission of a THz pulse. The THz pulse is directed by means of the collimator in the cell 122 through the gaseous biological sample to be studied to form a THz excitation beam.

An example of a THz excitation beam is shown in [FIG. 1B] (left diagram). It comprises, for example, electromagnetic pulses 10 emitted periodically with a period T; it is characterized by a spectrum 11 (right diagram) consisting of a frequency comb. The pulses 10 have a duration comprised between several hundred femtoseconds and a few picoseconds. The laser repetition frequency (equal to 1/T) can range from 1 GHz to a frequency of less than 1 Hz.

After traversing the sample, each THz pulse is directed to the antenna of the THz beacon 128 (receiver) by means of the collimator of the THz beacon 128 to form a THz sample beam which results from the convolution of the THz excitation beam with a transfer function associated with the gaseous biological sample and which depends on the sample characteristic parameters, especially its absorption.

The electric field of the terahertz pulses is measured at the antenna of the THz beacon 128 simultaneously illuminated by the optical probe pulse transported on the antenna of the THz beacon 128, for example, by means of an optical fiber 129, said optical probe pulse having been delayed with respect to the optical pulse sent to the antenna of the THz beacon 126 (emitter) by the delay line of the electromagnetic emission means 124. The electrical signal generated at the antenna can be amplified, then detected by electrical detection means 125. Electrical detection means 125 thus measure an electric field based on time, on scales ranging from femtoseconds to several hundred picoseconds or even of nanoseconds. A coherent detection of the THz sample beam is thus obtained.

Thus, as illustrated in [FIG. 1C], the time-dependent measurement can be performed by means of photoconductive or electro-optical sampling, as explained, for example, in [Ref. 4]. The delay line comprises, for example, mirrors mounted on a motorized translation deck, introducing a delay having a maximum time excursion tmax. Time sampling can also be performed by beating two frequency combs, the repetition rates of which are slightly different (so-called HASSOPS technique). The time interval between two measurements is referred to as sampling time ts, with sampling frequency fₛ=1/tₛ. The time excursion tₘₐₓ is the time range over which the measurement of the electric field is performed; said measurement of the electric field based on time is referred to as sample time trace Es(t). A typical sampling period tₛ is between about 10 and 50 fs. It depends on the cadence at which the femtosecond laser sends out optical pulses. The ability to directly measure the electric field of the THz pulse rather than the averaged energy gives access to both the phase and the amplitude of the waveform, and thus provides information on the absorption coefficient and the refractive index of the sample. If appropriate, said measurement of the electric field based on time is referred to as time trace Eref(t) under reference conditions, for example, under the same experimental conditions, but without the sample.

[FIG. 1D] exemplifies examples of a sample time trace Es(t) and a reference time trace Eref(t) detected in this way.

The processing unit 130 receives the time traces generated by the electrical detection means 125.

The processing unit 130 may comprise one or more computers or computing units. More generally, in the present description, whenever reference is made to calculation or processing steps for the implementation of method steps in particular, it is understood that each calculation or processing step can be implemented by software, hardware, firmware, microcode or any appropriate combination of these technologies. When software is used, each calculation or processing step can be implemented by computer program instructions or software code. These instructions can be stored in or transmitted to a computer-readable storage medium (or computing unit) and/or executed by a computer (or computing unit) in order to implement these calculation or processing steps.

The processing unit communicates with the TDS 120 to send requests and retrieve time traces in order to record them and process them. The processing unit can also interact with the set 140 comprising microcontroller(s) and/or sensor(s). For example, a microcontroller can allow the automation of a valve circuit (see [FIG. 2A]) and/or have an action to control the vacuum pump 150. One or more sensors can measure various parameters (pressure in the cell, temperature, CO₂ in the breath of the patient, etc.). The processing unit can then send requests to the sensors or a setpoint to the microcontroller and the values measured by the sensors can be sent to the processing unit.

[FIG. 2A] exemplifies in more detail and according to an example, the operation of a system for analyzing a biological sample at the gas analysis cell 122.

In this example, the collecting device 110 comprises a breath collecting tube 112 and a carbon dioxide (CO₂) sensor 115 arranged, for example, at the tube inlet, configured to measure CO₂ in the breath of a patient. From a predetermined CO₂ threshold value, the processing unit can control the opening of a solenoid valve 212 arranged on the bypass capillary 111 to draw the breath towards a tubular enclosure 220 of the gas analysis cell 122. The tubular enclosure 220 is, for example, a stainless steel tube with fittings to connect the various gas inlets and outlets and the sensors.

A solenoid valve 214 can be provided to open until a pressure setpoint in the enclosure 220 is met.

An intermediate enclosure 215 can also be provided to collect the breath during sampling. This allows, for example, the collection of a fraction of the breath, for example, the entire alveolar fraction of the breath, that is, the air present in the lungs. A valve 212 allows the collection of the desired fraction of the breath in the intermediate enclosure 215.

Opening the valve 214 then allows the tubular enclosure 220 to be filled up to a pressure chosen for the analysis; for example, if a measurement is to be taken at 10 mbar, the tubular enclosure is only filled with 10 mbar of gas included in the intermediate enclosure 215.

When the drainage of the enclosure 220 is requested, a solenoid valve 222 can open to empty the enclosure 220.

A heating element 230, for example, a heating wire, can be provided to regulate the temperature of the cell. A pressure gauge can be provided to measure the pressure in the cell.

In the example of [FIG. 2A], the THz beacons (emitter 255, receiver 265) are placed in enclosures 250, 260, in front of windows 253, 263 respectively, for example, windows inclined at the Brewster angle. The windows 253, 263 are supported by supports which can be mechanically connected to the enclosure 220 by means of parts 254, 264, for example, clamps.

The enclosures 250 and 260, for example, Plexiglas boxes, can be configured to allow the atmosphere between the antennae of the THz beacons 255, 265 and the windows 253, 263 to be purged in order to dispose of the water present in the air which could disturb the measurement. Indeed, between the antenna of the THz beacon and the window, the beam propagates in the open air, which is naturally charged with water. Since water is visible in the THz band, it introduces a bias into the measurement signal. Thus, filling the enclosures 250 and 260 with an inert gas, for example, nitrogen which is invisible in the THz band, expels the air and water it contains to saturate the atmosphere in the enclosures with nitrogen and to generate an inert atmosphere.

[FIG. 2B] exemplifies another example of a collecting device 210 configured to receive a liquid sample, for example, a urine, saliva or perspiration sample.

Such a collecting device can be connected to a tubular enclosure 220 of a gas analysis cell 122 as depicted in [FIG. 2A], replacing elements 110, 111, 112, 115, 212, 214, 215.

The collecting device 210 comprises a test tube 211 fitted with a vacuum fitting (not shown), a needle valve 213 allowing the regulation of the flow which enters into the gas analysis cell, a manual valve 217 allowing the passage to the cell to be completely closed off.

In operation, a sample purification step can first be performed. For this, the sample can be frozen in the test tube using, for example, a liquid nitrogen bath, then vacuum is applied in the test tube, by virtue of the tubular enclosure 220 which is under vacuum. Thus, the air from the test tube is removed.

Once this stage is completed, the valves 213, 217 can be closed.

To take the sample, it is possible, for example, to open the manual valve 217 and to control the opening of the needle valve. Thus, the liquid sample present in test tube 211 is subjected to vacuum, vaporizes and is drawn into the tubular enclosure 220.

As the tubular enclosure 220 fills up, it is possible to monitor the pressure evolution in the tubular enclosure 220, and to close the valves 213, 217 once the quantity (measured in pressure) of gaseous biological sample to be measured has been reached.

[FIG. 3] shows diagrams illustrating an example of a terahertz beacon according to different views in an analysis system according to the present disclosure.

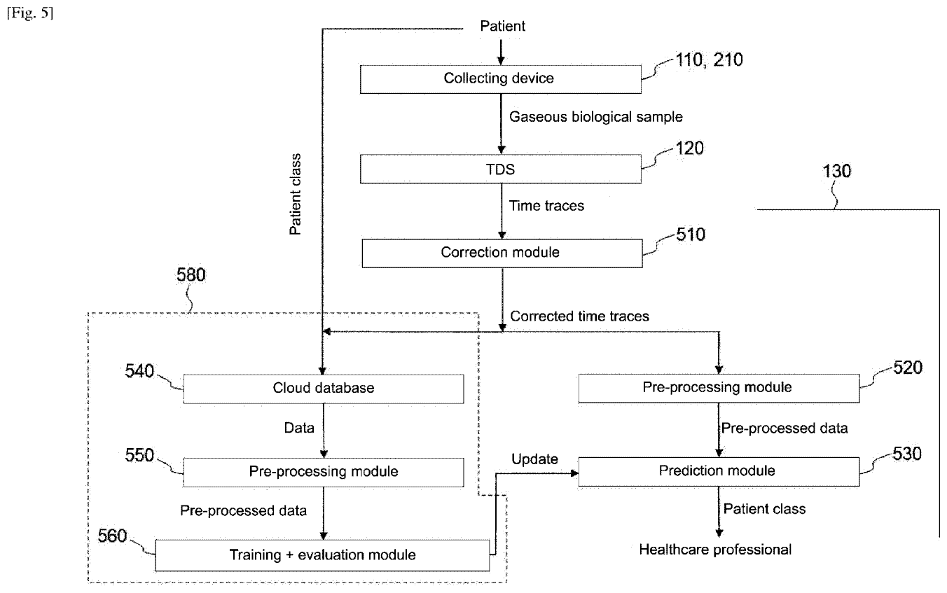

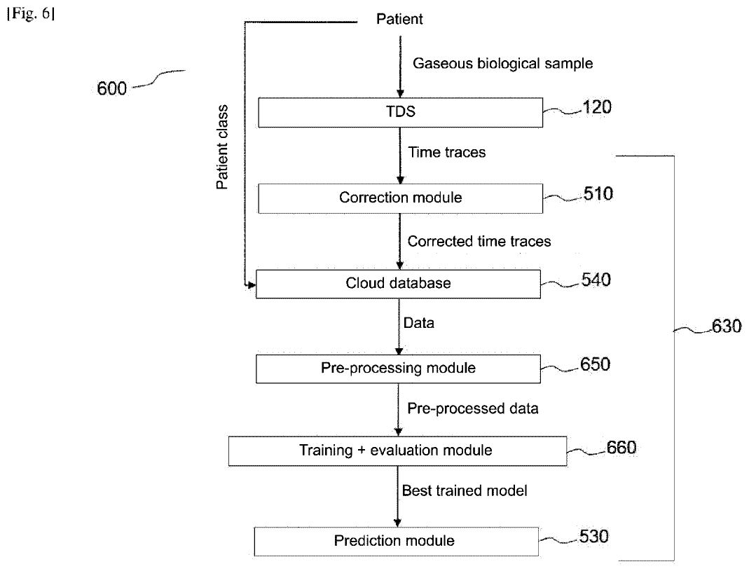

The THz beacon exemplified in [FIG. 3] comprises an antenna 310 and a plate 320 for adjusting the position and/or the orientation of the antenna 310, a collector 350, for example, a parabolic mirror, and a plate 360 for adjusting the position and/or the orientation of the parabolic mirror 350, a support part 370 to mechanically connect therebetween the antenna and parabolic mirror. In the example of [FIG. 3], the set formed by the antenna 310 and the plate 320 is attached to the support part 370 by means of a mechanical interface 330. In addition, a stand 380 attaches the THz beacon in the enclosure (250, 260, [FIG. 2A]) in which it must be attached.

[FIG. 4] shows a figure showing in a three-quarter view, a system for analyzing a gaseous biological sample according to the present disclosure, mounted in a rack 400. Especially, we can observe in [FIG. 4] the gas analysis cell 122, the device for collecting the gaseous biological sample, for example, a breath collecting device 110, the electromagnetic emission means 124 and the electrical detection means 125, the processing unit 130 and the vacuum pump 150.

[FIG. 5] is a block diagram exemplifying in more detail modules of a system for analyzing a gaseous biological sample according to the present disclosure, in implementation examples.

The gaseous biological sample of the patient is collected by a collecting device, for example a breath collecting device 110 as described by means of [FIG. 2A], or a collecting device 210 of a liquid sample as described by means of [FIG. 2B].

The gaseous biological sample is sent to the time-domain spectroscopy measurement device 120 to perform the THz spectroscopy measurement, for example, a device as described by means of [FIG. 1A] and [FIG. 2A]. The measured information is a time trace, that is, the measured electric field based on time.

In operation, a reference time trace can be associated with each measured sample time trace. The reference time trace is measured when there is no sample in the cell. The reference time trace can be measured periodically (for example, weekly, daily or between each patient).

For the same sample/reference, a set of time traces can be collected in order to increase the signal/noise ratio.

Time traces can be corrected by means of a correction module 510 in order to further increase the signal/noise ratio. An example of correction is a rectification of the offset of the traces relative to each other induced by the measurement system. An average can be made over all the time traces, after correction. For example, between about 500 time traces and about 1500 time traces, for example, about 1000 time traces, are recovered per sample, and per reference.

In operation, the patient and reference time traces can then be processed by a pre-processing module 520 in order to put them into the appropriate format for interpretation by a prediction module 530, also referred to as a machine learning module in the present disclosure. A sample estimator is then obtained from which the prediction module 530 can predict the classification of the patient (for example, sick patient or healthy patient, or assessment of the risk of developing a disease as a percentage, etc.). The classification is then processed by a healthcare professional.

A system for analyzing a gaseous biological sample, as exemplified in [FIG. 4], can thus be made available to a user for clinical use.

[FIG. 6] exemplifies a system 600 configured for pre-training the prediction module 530 of the processing unit of an analysis system according to the present disclosure.

The system can comprise all the elements of an analysis system according to the present disclosure; only the processing unit 630 is specifically configured to drive the prediction module 530.

Thus, the system 600 comprises a collecting device (not exemplified in [FIG. 6]) to generate a gaseous biological sample for a given patient and a time-domain spectroscopy measurement device 120 to perform the THz spectroscopy measurement, for example, a device as described by means of [FIG. 1A] and [FIG. 2A]. A plurality of time traces are generated, as described previously, and can be corrected by means of the correction module 510.

In parallel with these measurements, the patient takes a test depending on the disease being studied, in order to obtain information on their classification (for example, sick patient or healthy patient, type 1 or 2 diabetes; in the case of long-term cohort follow-up, development of a targeted disease, etc.). Thus, there is an associated known classification for each sample.

The performed measurements will allow the creation of the database 540 to be initiated. This will advantageously be a database recorded in electronic form, that is, in the Cloud. The database comprises, for example and in a non-limiting manner, for each patient, a unique identification, specific data (age, gender, etc.), the time traces of the measured sample for the patient, the reference time traces, the classification (sick patient or healthy patient, type 1 or 2 diabetes, time of the developed pathology, etc.).

Once the database has been created, the data can be sent to a pre-processing module 650. Its role is to prepare data that will be entered into a training and evaluation module 660. Data preparation allows information to be concentrated in order to improve predictions [Ref. 6].

Training consists of making the model learn from the data, that is, finding the parameters allowing the model to have good prediction performance criteria on the data. Several models (multilayer perceptron, recurrent neural network, etc.) [Ref. 7][Ref. 8] can be trained and then evaluated in order to select the one with the best performance. A model is a mathematical function with parameters that takes a data input and returns an output. Several types of models exist in the literature [Ref. 8][Ref. 9]. An example of a model that can be tested is a convolutional neural network, for example, a neural network capable of learning dependencies over long sequences or time series, or a “Long Short Term memory Neural Network” [Ref. 10].

The evaluation phase comes after the training; the different models can be compared using metrics (accuracy, false positive rate, false negative rate, etc.) [Ref. 11], depending on the risk of the studied disease. An example of a metric is recall which measures the rate of well-predicted positive cases among the positive cases for the studied disease. In fact during clinical use, false-positive results can be eliminated in subsequent steps after the control test.

Finally, the best trained model will be put into production and made accessible via a final interface between the prototype and the user to constitute the prediction module 530 ([FIG. 5]). Via this interface, the user can analyze the breath of the patient and have their classification.

In exemplary embodiments, if, during clinical use, the patient classification was previously known, their data can be fed into the database, as exemplified with reference to [FIG. 5]. The new data will then be used to improve the model and update the prediction module.

Thus, in exemplary embodiments, the system for analyzing a gaseous biological sample further comprises a module 580 for updating the prediction module 530.

In these exemplary embodiments, the measurements made during the clinical study can enrich the previously constituted database 540 to train the prediction model 530, for example, a database recorded in electronic form.

The database 540 is accessible by the processing unit 130 of the analysis system to send the new collected data there. As previously disclosed, the database 540 can contain patient information: a unique identification per patient, specific data (age, gender, etc.), the time traces Eref(t), Es(t), associated with the patient, the classification (sick or not sick; type 1 or 2 diabetes; etc.). The classification depends on the studied disease. Eref(t) and Es(t) can be obtained, for example, from the average of the time traces as previously explained.

Data arising from the database 540 is sent to a pre-processing module 550. As explained with reference to [FIG. 6], its role is to prepare the data that will be used during the training phase by a training and evaluation module 560 to improve predictions.

Training, as described in reference to [FIG. 6], consists of making the model learn from the data, that is, finding the parameters allowing the model to have good performance criteria on the data. An example of model that can be tested is the convolutional neural network. The evaluation phase, as previously explained, allows different models to be compared based on the risk of the studied disease.

At the output of the training and evaluation module, information on the patient classification is generated. It is then possible to update the prediction module 530 of the analysis system.

Although described by way of a number of detailed exemplary embodiments, the analysis systems comprise various variants, modifications and improvements which will be obvious to those skilled in the art, it being understood that these various variants, modifications and improvements form part of the scope of the invention, as defined by the following claims.

### REFERENCES

Ref 1: A. W. Boots, et al. “Exhaled molecular fingerprinting in diagnosis and monitoring: validating volatile promises”, Trends in Molecular Medicine, 21(10): 633-644, 2015.
Ref 2: A. Ulanowska et al. “The application of statistical methods using VOCs to identify patients with lung cancer”, Journal of Breath Research, 5(4): 046008, 2011.
Ref.3: M. Phillips et al. “Variation in volatile organic compounds in the breath of normal humans”, Journal of Chromatography B: Biomedical Sciences and Applications, 729(1-2): 75-88, 1999.
Ref. 4: EP 3 865 857
Ref. 5: Y. Kistenev et al. “Diagnosis of Diabetes Based on Analysis of Exhaled Air by Terahertz Spectroscopy and Machine Larning”, Optics and Spectroscopy, 128(6), pp. 809-814, 2020.
Ref. 6: Benhar, H., Idri, A., & Fernândez-Alemân, J. L. (2020). Data preprocessing for heart disease classification: A systematic literature review. Computer Methods and Programs in Biomedicine, 195, 105635.
Ref. 7 Kotsiantis, S. B., Zaharakis, I. D., & Pintelas, P. E. (2006). Machine learning: a review of classification and combining techniques. Artificial Intelligence Review, 26(3), 159-190.
Ref. 8 Craik, A., He, Y., & Contreras-Vidal, J. L. (2019). Deep learning for electroencephalogram (EEG) classification tasks: a review. Journal of neural engineering, 16(3), 031001.
Ref. 9 Kiranyaz, S., Ince, T., Abdeljaber, O., Avci, O., & Gabbouj, M. (2019, May). 1-D convolutional neural networks for signal processing applications. In ICASSP 2019-2019 IEEE International Conference on Acoustics, Speech and Signal Processing (ICASSP) (pp. 8360-8364). IEEE.
Ref. 10 Murat, F., Yildirim, O., Talo, M., Baloglu, U. B., Demir, Y., & Acharya, U. R. (2020). Application of deep learning techniques for heartbeats detection using ECG signals-analysis and review. Computers in biology and medicine, 120, 103726.
Ref. 11 Hossin, M., & Sulaiman, M. N. (2015). A review on evaluation metrics for data classification evaluations. International journal of data mining & knowledge management process, 5(2), 1.

1-10. (canceled)

11. A system for analyzing a gaseous biological sample comprising:
a collecting device for collecting said gaseous biological sample;
a time-domain spectroscopy measurement device comprising:
a gas analysis cell configured to receive the collected gaseous biological sample;
electromagnetic emission means configured to emit a substantially collimated THz excitation beam into the gas analysis cell;
electromagnetic detection means configured to detect at least one first sample time trace, each sample time trace resulting from a coherent detection of a THz sample beam arising from the gas analysis cell traversed by the THz excitation beam;
a processing unit comprising a pre-trained machine learning module for detecting at least one state of a subject, the processing unit being configured to:
calculate a sample estimator on the basis of said at least one sample time trace;
determine from said estimator and using the machine learning module, said at least one state of the subject.

12. The system for analyzing a gaseous biological sample according to claim 11, wherein
the time-domain spectroscopy measurement device is configured to further detect at least one first reference time trace; and
said sample estimator is calculated on the basis of said at least one sample time trace and said at least one reference time trace.

13. The system for analyzing a gaseous biological sample according to claim 11, wherein the detection means are configured to detect a plurality of sample time traces, the sample estimator being calculated on the basis of said plurality of sample time traces.

14. The system for analyzing a gaseous biological sample according to claim 11, wherein the machine learning module is pre-trained by means of a convolutional neural network.

15. The system for analyzing a gaseous biological sample according to claim 11, wherein the collecting device is configured to collect the breath of a patient and comprises a breath collecting tube.

16. The system for analyzing a gaseous biological sample according to claim 15, wherein the collecting device further comprises carbon dioxide detection means configured to detect the carbon dioxide in the collected breath, a first solenoid valve and first solenoid valve control means configured to open the solenoid valve when the detected carbon dioxide exceeds a predetermined threshold value, the collected breath can then be drawn towards the gas analysis cell.

17. The system for analyzing a gaseous biological sample according to claim 15, wherein the collecting device further comprises an intermediate enclosure configured to receive at least one fraction of the collected breath, a second solenoid valve and second solenoid valve control means configured to open said second solenoid valve, the collected breath can then be drawn towards the gas analysis cell and close said second solenoid valve when a pressure in the gas analysis cell reaches a predetermined threshold value.

18. The system for analyzing a gaseous biological sample according to claim 11, wherein the collecting device is configured to collect a liquid biological sample and comprises production means for producing, from the liquid biological sample, a gaseous biological sample.

19. The system for analyzing a gaseous biological sample according to claim 11, wherein the electromagnetic emission means and the electromagnetic detection means each comprise a THz beacon (255, 265), each of the THz beacons comprising an antenna, a deflecting mirror, for example, a parabolic mirror, and a support configured to securely hold said antenna and said parabolic mirror.

20. The system for analyzing a gaseous biological sample according to claim 11, further comprising:
a database comprising, for a set of subjects, at least one sample time trace associated with a state of the subject, and
a training module for training the machine learning module from data in the database;
the processing unit being further configured to send said at least one sample time trace generated by the time-domain spectroscopy measurement device to said database.
